(19) United States
(12) Patent Application Publication
(10) Pub. No.: US 2026/0358259 A1
(43) Pub. Date: Nov. 12, 2026

(54) SLAB-BASED OPTICAL DEVICE, SYSTEM, AND METHOD TO CAPTURE IMAGE OF TEST DEVICE

(71) Applicant: INITO HEALTH INC., Houston, TX (US)

(72) Inventors: Varun AKUR VENKATESAN, Bangalore (IN); Gautam SHARMA, Bangalore (IN); Siraj ISSANI, Bangalore (IN)

(21) Appl. No.: 19/486,976

(22) PCT Filed: May 24, 2024
(86) PCT No.: PCT/IN2024/050613

§ 371 Date: Nov. 24, 2025

(30) Foreign Application Priority Data
May 24, 2023 (IN) 202241067794

(51) Int. Cl. G01N 21/77; G01N 21/01; G01N 33/543

(52) U.S. Cl. CPC G01N 21/77; G01N 21/01; G01N 33/54388; G01N 2021/7759

(57) ABSTRACT

Disclosed is an imaging device 10 includes one or more light sources 1A-1N configured to illuminate an object 5 and one or more reflector elements 2A-2N configured to direct luminescence towards the object 5. Further, disclosed is a slab-based optical device 100 includes a first illumination device 102A and a second illumination device 102B, a first slab 104A and a second slab 104B adapted to receive the luminescence from the first illumination device 102A and the second illumination device 102B, a test strip 106 removably disposed between the first slab 104A and the second slab 104B and an image sensor 108 adapted to capture one or more images of the test strip 106. Further, a method 400 to determine one or more images captured by a slab-based optical device 100 is provided.

### TECHNICAL FIELD

The present aspect relates generally to a medical device and more particularly to slab-based optical device, system, and method for capturing image(s) from a test device.

### BACKGROUND

Test devices such as test strips are an apparatus that are used to measure any concentration of a chemical or some characteristics of a liquid.

Test strips are a simple, effective analytical tool used for the rapid detection of an analyte in the field or on-site. These test devices are subjected to detect any analyte and further generate a result on the test device. Such generated result is either visualized and analyzed by naked eye or by an optical detection device.

The results that are visualized by naked eye are qualitative and it provides the presence or absence of an analyte or any chemicals in the bodily fluids. The results that are visualized by optical detection device may be quantitative. The major limitations' associated with the conventional optical detection device are the false results due to the poor illumination. Such poor luminescence diminishes the picture of results in the test strip. The poor illumination also reduces the brightness, contrast, hue, saturation in the captured image. The camera may also flicker while capturing the image due to the poor luminescence, that results in failure in the quantitative analysis.

In some scenario due to the direct light that falls on the detection band may emit bright light after getting scattered from the liquid molecules on the track of the test strip. Such scattering of light may result in inaccurate results in quantitative analysis.

Thus, there is a need for a technical solution that overcomes the aforementioned problems of conventional optical device that have poor illumination to perform quantitative analysis on a result detected in the test strip.

### SUMMARY OF INVENTION

In an aspect, an imaging device is provided. The imaging device includes one or more light sources configured to illuminate an object and one or more reflector elements configured to direct luminescence towards the object.

In some aspects, the object is one or more test strips adapted to receive a biological sample.

In some aspects, the one or more reflector elements further includes a first face configured to receive light imparted by one or more light sources, a second face configured to exit the light received from the first face and a third face configured to receive the reflected light and form an image.

In some aspects, the light illuminated by one or more light sources penetrates into the first face of the one or more reflector elements and exits through the second face of the one or more reflector elements to illuminate the object.

In some aspects, the light reflected back from the object penetrates the second face of one or more reflector elements and incidences on the third face of the one or more reflector elements to form the image.

In some aspects, the imaging device further includes an imaging sensor adapted to capture one or more images of the object formed on the third face of the one or more reflector elements.

In some aspects, the imaging sensor captures one or more images of a first site and second site of the object.

In some aspects, the imaging sensor captures the one or more images of a first zone of interest positioned at the first side of the object through the third face and a second zone of interest positioned at the second side of the object through the third surface.

In some aspects, the one or more light sources is selected from a group comprising LED, backlight, light beam or organic light emitting diode.

In some aspects, the one or more reflector elements may be positioned between the one or more light sources device and the object.

In some aspects, the imaging device further includes one or more lens positioned between the one or more reflector elements and the imaging sensor.

In another aspect, a slab-based optical device is provided. The slab-based optical device includes a first illumination device and a second illumination device, a first slab and a second slab adapted to receive the luminescence from the first illumination device and the second illumination device, a test strip removably disposed between the first slab and the second slab, the test strip adapted to receive the refracted luminescence from the first slab and a second slab, an image sensor adapted to capture one or more images of the test strip.

In some aspects, the first slab further incudes a first face, a second face, a third face such that the first luminescence received through the first face exits from the second face of the first slab.

In some aspects, the first luminescence entering the first face of the first slab is greater than the critical angle of the first slab.

In some aspects, the second slab further comprises a first surface, a second surface, and a third surface, such that the second luminescence received through the first surface exits from the second face of the second slab.

In some aspects, the luminescence entering the first surface of the second slab is greater than a critical angle of the second slab.

In some aspects, the slab-based optical device further includes one or more lens disposed adjacent to the imaging device.

In another aspect of the present disclosure, a method of working of the slab-based optical device is provided. The method includes illuminating luminescence from a first and second illumination device, passing the illuminated light through a first slab and slab respectively, reflecting light on a first zone and second zone of interest disposed on a test strip, and capturing one or more images of the first zone and second zone of interest disposed on a first and second side of the test strip.

In another aspect, a slab-based optical device is provided. The device includes a first illumination device and a second illumination device adapted to provide first luminescence and second luminescence, respectively. A first slab that is disposed below the first illumination device and is adapted to receive the first luminescence, a second slab that is disposed below the second illumination device and is adapted to receive the second luminescence. A test strip that is removably disposed between the first slab and the second slab and adapted to receive the first luminescence and the second luminescence, respectively. A first lens disposed adjacent to an image sensor and adapted to magnify one or more image and the imaging device adapted to capture one or more images of the test strip.

In another aspect, a slab-based optical device is provided. The device includes a first illumination device and a second illumination device adapted to provide first luminescence and second luminescence, respectively. A first slab that is disposed below the first illumination device and is adapted to receive the first luminescence, a second slab that is disposed below the second illumination device and is adapted to receive the second luminescence. A test strip that is removably disposed between the first slab and the second slab and adapted to receive the first luminescence and the second luminescence, respectively. One or more lens disposed between the first slab, second slab and an image sensor and further adapted to magnify one or more images formed. The imaging device is adapted to capture one or more images of the test strip.

### BRIEF DESCRIPTION OF DRAWINGS

The above and still further features and advantages of aspects of the present disclosure becomes apparent upon consideration of the following detailed description of aspects thereof, especially when taken in conjunction with the accompanying drawings, and wherein:

FIG. 1 illustrates an imaging device, in accordance with an aspect of the present disclosure;

FIG. 2 illustrates a front view of a slab-based optical device, in accordance with an aspect of the present disclosure;

FIG. 3 illustrates a front view of an exemplary slab-based optical device, in accordance with an aspect of the present disclosure;

FIG. 4 illustrates a front view of an exemplary slab-based optical device, in accordance with an aspect of the present disclosure; and

FIG. 5 illustrates a flowchart that depicts method of working of the slab-based optical device of FIG. 2, in accordance with an aspect of the present disclosure.

To facilitate understanding, like reference numerals have been used, where possible, to designate like elements common to the figures.

### DETAILED DESCRIPTION

Various aspect of the present disclosure provides a slab-based optical device, system and method to capture image of a test device. The following description provides specific details of certain aspects of the disclosure illustrated in the drawings to provide a thorough understanding of those aspects. It should be recognized, however, that the present disclosure can be reflected in additional aspects and the disclosure may be practiced without some of the details in the following description.

The various aspects including the example aspects are now described more fully with reference to the accompanying drawings, in which the various aspects of the disclosure are shown. The disclosure may, however, be embodied in different forms and should not be construed as limited to the aspects set forth herein. Rather, these aspects are provided so that this disclosure is thorough and complete, and fully conveys the scope of the disclosure to those skilled in the art. In the drawings, the sizes of components may be exaggerated for clarity.

It is understood that when an element or layer is referred to as being “on,” “connected to,” or “coupled to” another element or layer, it can be directly on, connected to, or coupled to the other element or layer or intervening elements or layers that may be present. As used herein, the term “and/or” includes any and all combinations of one or more of the associated listed items.

Spatially relative terms, such as “top,” “bottom,” and the like, may be used herein for ease of description to describe one element or feature's relationship to another element(s) or feature(s) as illustrated in the figures. It is to be understood that the spatially relative terms are intended to encompass different orientations of the structure in use or operation in addition to the orientation depicted in the figures.

The subject matter of example aspects, as disclosed herein, is described with specificity to meet statutory requirements. However, the description itself is not intended to limit the scope of this patent. Rather, the inventor/inventors have contemplated that the claimed subject matter might also be embodied in other ways, to include different features or combinations of features similar to the ones described in this document, in conjunction with other technologies. Generally, the various aspects including the example aspects relate to an imaging device, a slab-based optical device and method to capture image of the test device have a single inventive concept that includes an optical based imaging device for capturing one or more images of the object/test strip having a biological sample and further determine concentration of one or more analytes present in the biological sample.

As mentioned, there remains a need for an optical device that overcomes the limitations associated with the available optical devices such as poor illumination, scattering of light due to the presence of glossy and liquid molecules. The present disclosure provides a slab-based optical device, system, and method that provides slab based illumination to a test strip that enables an imaging device to capture one or more images of results obtained at the test strip in an enlarged perspective. The present disclosure also provides slab-based illumination device that prevents scattering of light on the test strip. The present disclosure also provides slab-based illumination device that deliver more accurate and errorless quantitative results compared to the available optical devices.

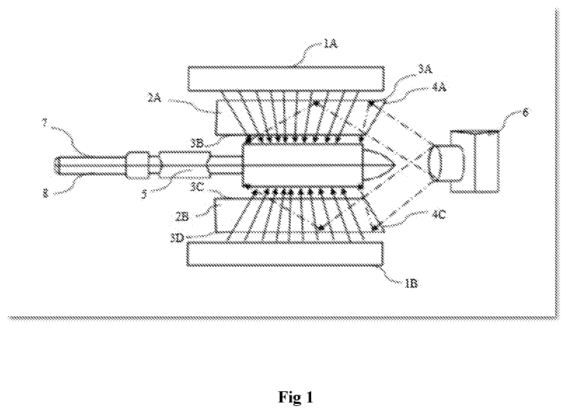

FIG. 1 illustrates an imaging device 10, in accordance with an aspect of the present disclosure.

The imaging device 10 may include one or more light sources 1A-1N of which first through second light sources 1A-1B are explicitly shown, one or more reflector elements 2A-2N of which first through second reflector elements 2A-2B are explicitly shown, an object 5 and an imaging sensor 6.

In some aspects, the one or more light sources 1A-IN may be adapted to provide luminescence to the imaging device 10. In some aspects of the present disclosure, the one or more light sources 1A-1N may include, but not limited to, a visible light, an infrared light, an ultraviolet light and the like. Aspects of the present disclosure are intended to include or otherwise cover any type of light source, including known, related art, and/or later developed technologies.

In some aspects, the one or more reflector elements 2A-2N may be disposed below the one or more light sources 1A-1N. In some aspects, the one or more reflector elements 2A-2N may include, but not limited to, a glass, quartz, sapphire, acrylate, polycarbonate and the like. Aspects of the present disclosure are intended to include or otherwise cover any type of reflector elements, including known, related art, and/or later developed technologies.

In some aspects, the first reflector element 2A and the second reflector element 2B may be disposed below the first light source 1A and the second light source 1B, respectively. The first reflector element 2A may include a first face 3A, a second face 3B and a third face 4A.

The first face 3A of the first reflector element 2A may be adapted to receive the luminescence from the first light source 1A. In some aspects of the present disclosure, the luminescence entering the first face 3A of the first reflector element 2A may be greater than a critical angle of the first reflector element 2A such that incidence luminescence undergoes refraction and penetrates the first face 3A of the first reflector element 2A.

The second face 3B may be other side of the first face 3A of the first reflector element 2A. The luminescence passes through the first face 3A may be adapted to exit from the second face 3B of the first reflector element 2A. Specifically, the incidence luminescence penetrates the first face 3A, undergoes further refraction and exits the first reflector element 2A through the second face 3B. The third face 4A may be disposed on either sides of the first reflector element 2A.

In some aspects, the second reflector element 2B may be disposed above the second light source 1B. The second reflector element 2B may include a first surface 3C, a second surface 3D and a third surface 4B.

The first surface 3C of the second reflector element 2B may be adapted to receive the luminescence from the second light source 1B. In some aspects of the present disclosure, the luminescence entering the first surface 3C of the second reflector element 2B may be greater than a critical angle of the second reflector element 2B such that incidence luminescence undergoes refraction and penetrates the first surface 3C of the second reflector element 2B.

The second surface 3D may be other side of the first surface 3C of the second reflector element 2B. The luminescence passes through the first surface 3C may be adapted to exit from the second surface 3D of the second reflector element 2B. Specifically, the incidence luminescence penetrates the first surface 3C, undergoes further refraction and exits the second reflector element 2B through the second surface 3D. The third face 4B may be disposed on either sides of the second reflector element 2B.

In some aspects, the object 5 may be removably placed between the first reflector element 2A and the second reflector element 2B. In some aspects, the object 5 may be adapted to receive a biological sample. In some aspects, the biological sample may be selected from a group, but not limited to, blood, serum, plasma, urine, saliva, sweat and the like. Aspects of the present disclosure are intended to include or otherwise cover any type of biological sample, including known, related art, and/or later developed technologies. In some aspects, the object may include, but not limited to, a test strip, a lateral flow assay, assay strips, reagent strip and the like. Aspects of the present disclosure are intended to include or otherwise cover any type of objects, including known, related art, and/or later developed technologies. In some aspects, the object 5 may be a test strip adapted to receive a biological sample.

In some aspects, the object 6 may be adapted to receive the refracted luminescence from the second face 3B of the first reflector element 3A and the second surface 3D of the second reflector element 2B.

In some aspects, the object 6 may include a first side 7 and a second side 8.

The first side 7 may be adjacent to the first reflector element 2A. The first side 7 may include a first zone of interest, where a first set of results are detected. The second side 8 may be adjacent to the second reflector element 2B. The second side 8 may include a second zone of interest, where a second set of results is detected. In some aspects of the present disclosure, the first reflector element 2A and the second slab 2B may be adapted to magnify the first zone of interest and the second zone of interest.

The imaging sensor 6 may be disposed adjacent to the third face 4A of the first reflector element 2A and the third surface 4B of the second reflector element 2B. The imaging sensor 6 may be adapted to capture one or more images of a first zone of interest positioned at the first side 7 of the object 6 through the third face 4A and a second zone of interest positioned at the second side 8 of the object 106 through the third surface 4B. In some aspects, the imaging device may include one or more imaging sensors to capture one or more images of the first zone of interest positioned at the first side 7 of the object 6 and the second zone of interest positioned at the second side 8 of the object 106.

In some aspects, one or more lens (not shown) may be positioned in between the one or more reflector elements 2A-2N and the imaging sensor 6.

In some other aspects of the present disclosure, the one or more captured images may further be analysed to determine one or more body parameters of the user by way of a determination system (not shown). The determination system may be adapted to receive the one or more captured images from the imaging sensor 6. The determination system may be adapted to analyse the detected results in the captured one or more images by comparing the captured one or more images with a predetermined images or a predetermined threshold stored in a storage unit (not shown).

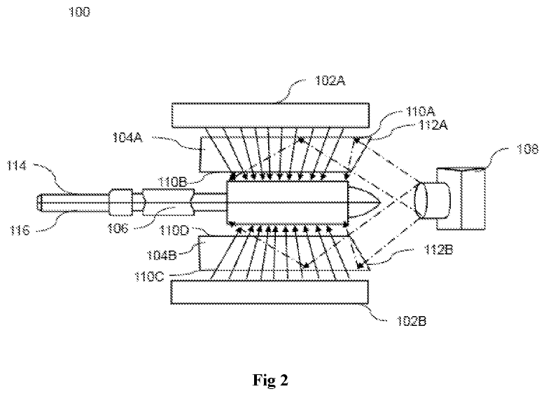

FIG. 2 illustrates a front view of a slab-based optical device 100, in accordance with an aspect of the present disclosure.

The slab-based optical device 100 may include a first illumination device 102A a second illumination device 102B, a first slab 104A, a second slab 104B, a test strip 106, and an image sensor 108.

The first illumination device 102A and the second illumination device 102B may be adapted to provide luminescence to the slab-based optical device 100. In some aspects of the present disclosure, either the first illumination device 102A and the second illumination device 102B may include, but not limited to, a visible light, an infrared light, an ultraviolet light and the like. Aspects of the present disclosure are intended to include or otherwise cover any type of light source, including known, related art, and/or later developed technologies.

The first slab 104A may be disposed below the first illumination device 102A. The first slab 104A may be adapted to receive the luminescence produced by the first illumination device 102A.

The first slab 104A may include a first face 110A, a second face 110B, and a third face 112A.

The first face 110A of the first slab 104A may be adapted to receive the luminescence from the first illumination device 102A. In some aspects of the present disclosure, the luminescence entering the first face 110A of the first slab 104A may be greater than a critical angle of the first slab 104A. Such that incidence luminescence undergoes refraction and penetrates the first face 110A of the first slab 104A.

The second face 110B may be other side of the first face 110A of the first slab 104A. The luminescence passes through the first face 110A may be adapted to exit from the second face 110B of the first slab 104A. Specifically, the incidence luminescence penetrates the first face 110A, undergoes further refraction and exits the first slab 104A through the second face 110B.

The third face 112A may be disposed on either side of the first slab 104.

The second slab 104B may be disposed above the second illumination device 102B. The second slab 104B may be adapted to receive the luminescence produced by the second illumination device 102B.

The second slab 104B may include a first surface 110C, a second surface 110D, and a third surface 112B.

The first surface 110C of the second slab 104B may be adapted to receive the luminescence from the second illumination device 102B. In some aspects of the present disclosure, the luminescence entering the first surface 110C of the second slab 104B may be greater than a critical angle of the second slab 104B. Such that incidence luminescence undergoes refraction and penetrates the first surface 110C of the second slab 104B.

The second surface 110D may be other side of the first surface 110C of the second slab 104B. The luminescence passes through the first surface 110C may be adapted to exit from the second surface 110D of the second slab 104B. Specifically, the incidence luminescence penetrates the first surface 110C, undergoes further refraction and exits the second slab 104B through the second surface 110D.

The test strip 106 may be removably placed between the first slab 104A and the second slab 104B. The test strip 106 may be adapted to receive the refracted luminescence from the second face 110B of the first slab 104A and the second surface 110D of the second slab 104B (as shown in line arrows in FIG. 1).

The test strip 106 may include a first side 114 and a second side 116.

The first side 114 may be adjacent to the first slab 104A. The first side 114 may include a first zone of interest, where a first set of results are detected.

The second side 116 may be adjacent to the second slab 104B. The second side 116 may include a second zone of interest, where a second set of results are detected. In some aspects of the present disclosure, the first slab 104A and the second slab 104B may be adapted to magnify the first zone of interest and the second zone of interest.

The refracted luminescence from the first slab 104A and the second slab 104B may be adapted to project on the first zone of interest in the first side 114 of the test strip 106 and the second zone of interest in the second side 116 of the test strip 106 respectively. The image sensor 108 may be disposed adjacent to the third face 112A of the first slab 104A and the third surface 112B of the second slab 104B.

The image sensor 108 may be adapted to capture one or more images of the first zone of interest in the first side 114 of the test strip 106 through the third face 112A and the second zone of interest in the second side 114 of the test strip 106 through the third surface 112B. In some aspects, the image sensor 108 may capture one or more images from either sides of the test strip 106 to provide accurate concentration of one or more analytes present in the biological sample received on the test strip 106. In some aspects, the slab-based optical device 100 may be adapted to capture one or more images of one or more tracks (not shown) positioned on the first side 114 and second site 116 of the test strip 106, respectively.

In some aspects, the slab-based optical device 100 may include one or more lens (not shown) disposed adjacent to the image sensor 108.

In an exemplary aspect, the slab-based optical device 100 may be adapted to capture one or more images of one or more test strips (not shown).

In some other aspects of the present disclosure, the one or more captured images may further be analysed to determine one or more body parameters of the user by way of a determination system (not shown). The determination system may be adapted to receive the one or more captured images from the imaging device from the image sensor 108. The determination system may be adapted to analyse the detected results in the captured one or more images by comparing the captured one or more images with a predetermined images or a predetermined threshold stored in a storage unit (not shown).

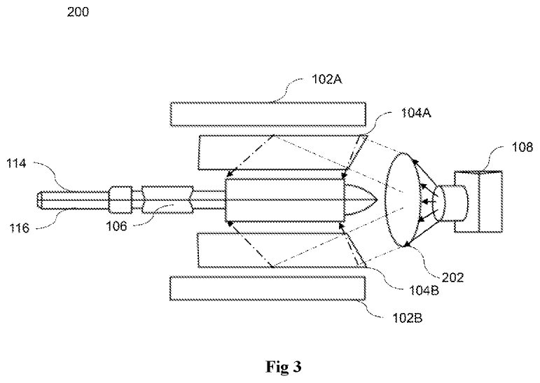

FIG. 3 illustrates a front view of an exemplary slab-based optical device 200, in accordance with an aspect of the present disclosure.

The slab-based optical device 200 is substantially similar to that of the slab-based optical device 100 of FIG. 2 with like elements referred by way of like reference numerals. However, the addition of one lens between the image sensor 108 and the first slab 104A and the second slab 104B is different from the slab-based optical device 100. The slab-based optical device 200 may include a first lens 202 adapted to magnify the one or more formed images.

The first lens 202 may be disposed adjacent to the image sensor 108. In some aspects of the present disclosure, the first lens 202 may be disposed between the first slab 104A, the second slab 104B and the image sensor 108.

The image sensor 108 may be adapted to capture one or more images of the first zone of interest in the first side 114 of the test strip 106 through the first slab 104A and the first lens 302. The image sensor 108 may be adapted to capture one or more images of the second zone of interest in the second side 116 of the test strip 106 through the second slab 104B and the first lens 302.

In some other aspects of the present disclosure, the one or more captured images may further be analysed to determine the one or more body parameters of the user by way of the determination system (not shown). The determination system may be adapted to receive the one or more captured images of the results generated on the first zone of interest and the results generated on the second zone of interest from the imaging device from the image sensor 108. The determination system may be adapted to analyse the detected results in the captured one or more images by comparing the captured one or more images with the predetermined images or the predetermined threshold stored in the storage unit.

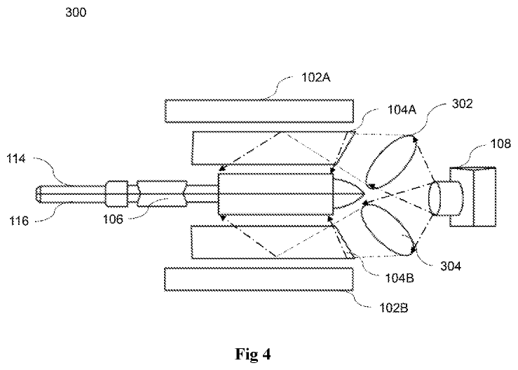

FIG. 4 illustrates a front view of an exemplary slab-based optical device 300, in accordance with an aspect of the present disclosure.

The slab-based optical device 300 is substantially similar to that of the slab-based optical device 100 of FIG. 2 with like elements referred by way of like reference numerals. However, the addition of one or more lens between the image sensor 108 and the first slab 104A and the second slab 204B is different from the slab-based optical device 100.

The slab-based optical device 300 may include a second lens 302 and a third lens 304 adapted to magnify the one or more formed images.

The second lens 302 may be substantially disposed adjacent to the image sensor 108. In some aspects of the present disclosure, the second lens 302 may be disposed between the first slab 104A and the image sensor 108.

The third lens 304 may be substantially disposed adjacent to the image sensor 108. In some aspects of the present disclosure, the third lens 304 may be disposed between the second slab 104B and the image sensor 108.

The image sensor 108 may be adapted to capture one or more images of the first zone of interest in the first side 114 of the test strip 106 through the first slab 104A and the second lens 302. The image sensor 108 may be adapted to capture one or more images of the second zone of interest in the second side 116 of the test strip 106 through the second slab 104B and the third lens 304.

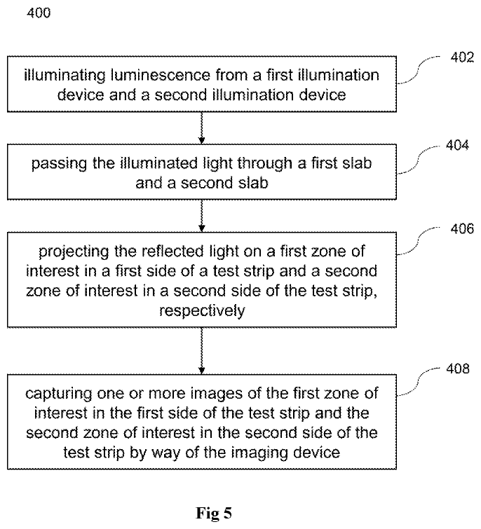

FIG. 5 illustrates a flowchart that depicts method 400 of working of the slab-based optical device 100, in accordance with an aspect of the present disclosure. The method includes

At step 402, the slab-based optical device 100 may be configured to illuminate luminescence from the first illumination device 102A and the second illumination device 102B.

At step 404, the slab-based optical device 100 may be adapted to pass the illuminated light through the first slab 104A and the second slab 104B.

At step 406, the slab-based optical device 100 may be adapted to project the reflected light on the first zone of interest in the first side 114 of the test strip 106 and the second zone of interest in the second side 116 of the test strip 106 respectively.

At step 408, the slab-based optical device 100 may be configured to capture one or more images of the first zone of interest in the first side 114 of the test strip 106 and the second zone of interest in the second side 116 of the test strip 106 by way of the image sensor 108.

### Advantages of the Present Device

1. The slab-based optical device of the present disclosure may capture one or more images of the zone of interest with high contrast and brightness.
2. The slab-based optical device of the present disclosure may provide quantitative results more accurately compared to the conventional optical devices.
3. The slab-based optical device of the present disclosure may capture one or more images of the zone of interest with high contrast and brightness.
4. The slab-based optical device of the present disclosure captures one or more images from either sides of the test strip and provides more accurate concentration values of analytes.
5. The slab-based optical device of the present disclosure enables the image sensor to capture image of the test strip without a light scatter due to the presence of glossy molecules.
6. The slab-based optical device of the present disclosure may enable a user to analyse multiple tracks on the test strips by capturing sides of the test device.

The foregoing discussion of the present disclosure has been presented for purposes of illustration and description. It is not intended to limit the present disclosure to the form or forms disclosed herein. In the foregoing Detailed Description, for example, various features of the present disclosure are grouped together in one or more aspects, configurations, or aspects for the purpose of streamlining the disclosure. The features of the aspects, configurations, or aspects may be combined in alternate aspects, configurations, or aspects other than those discussed above. This device of disclosure is not to be interpreted as reflecting an intention the present disclosure requires more features than are expressly recited in each claim. Rather, as the following claims reflect, inventive aspects lie in less than all features of a single foregoing disclosed aspect, configuration, or aspect. Thus, the following claims are hereby incorporated into this Detailed Description, with each claim standing on its own as a separate aspect of the present disclosure.

Moreover, though the description of the present disclosure has included description of one or more aspects, configurations, or aspects and certain variations and modifications, other variations, combinations, and modifications are within the scope of the present disclosure, e.g., as may be within the skill and knowledge of those in the art, after understanding the present disclosure. It is intended to obtain rights which include alternative aspects, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

I claim:

1. An imaging device 10, comprising:
a. one or more light sources 1A-1N configured to illuminate an object 5; and
b. one or more reflector elements 2A-2N configured to direct luminescence towards the object 5.

2. The imaging device 10 as claimed in claim 1, wherein the object 5 is one or more test strips adapted to receive a biological sample.

3. The imaging device 10 as claimed in claim 1, wherein the one or more reflector elements 2A-2N further comprises:
a. a first face 3A configured to receive light imparted by one or more light sources 1A-1N;
b. a second face 3B configured to exit the light received from the first face 3A; and
c. a third face 4A configured to receive the reflected light and form an image.

4. The imaging device 10 as claimed in claim 3, wherein the light illuminated by one or more light sources 1A-1N penetrates into the first face 3A of the one or more reflector elements 2A-2N and exits through the second face 3B of the one or more reflector elements 2A-2N to illuminate the object 5.

5. The imaging device 10 as claimed in claim 3, wherein the light reflected back from the object 5 penetrates the second face 3B of one or more reflector elements 2A-2N and incidences on the third face 4A of the one or more reflector elements 2A-2N to form the image.

6. The imaging device 10 as claimed in claim 1, wherein the imaging device 10 further comprises an imaging sensor 6 adapted to capture one or more images of the object 5 formed on the third face 4A of the one or more reflector elements 2A-2N.

7. The imaging device 10 as claimed in claim 6, wherein the imaging sensor 6 captures one or more images of a first site 7 and a second site 8 of the object 5.

8. The imaging device 10 as claimed in claim 7, wherein the imaging sensor 6 captures one or more images of a first zone of interest positioned at the first side 7 of the object 6 through the third face 4A and a second zone of interest positioned at the second side 8 of the object 5 through the third surface 4B.

9. The imaging device 10 as claimed in claim 1, wherein the one or more light sources 1A-1N is selected from a group comprising LED, backlight, light beam or organic light emitting diode.

10. The imaging device 10 as claimed in claim 1, wherein the one or more reflector elements 2A-2N is positioned between the one or more light sources 1A-1N device and the object 5.

11. The imaging device 10 as claimed in claim 1, further comprises one or more lens positioned between the one or more reflector elements 2A-2N and the imaging sensor 6.

12. A slab-based optical device 100 comprising:
a. a first illumination device 102A and a second illumination device 102B adapted to provide first luminescence and second luminescence, respectively;
b. a first slab 104A that is disposed below the first illumination device 102A, and is adapted to receive the first luminescence;
c. a second slab 104B that is disposed below the second illumination device 102B, and is adapted to receive the second luminescence;
d. a test strip 106 that is removably disposed between the first slab 104A and the second slab 104B, and adapted to receive the first luminescence and the second luminescence, respectively; and
e. an image sensor 108 adapted to capture one or more images of the test strip 106.

13. The slab-based optical device 100 as claimed in claim 12, wherein the first slab 104A further comprises a first face 110A, a second face 110B and a third face 112A such that the first luminescence received through the first face 110A exits from the second face 110B of the first slab 104A.

14. The slab-based optical device 100 as claimed in claim 12, wherein the first luminescence entering the first face 110A of the first slab 104A is greater than the critical angle of the first slab 104A.

15. The slab-based optical device 100 as claimed in claim 12, wherein the second slab 104B further comprises a first surface 110C, a second surface 110D, and a third surface 112B, such that the second luminescence received through the first surface 110c exits from the second face 110D of the second slab 104B.

16. The slab-based optical device 100 as claimed in claim 12, wherein the luminescence entering the first surface 110C of the second slab 104B is greater than a critical angle of the second slab 104B.

17. The slab-based optical device 100 as claimed in claim 12, further comprises one or more lens disposed adjacent to the image sensor 108.

18. A method 400 to determine one or more images captured by a slab-based optical device 100, comprising:
a. illuminating 402 luminescence from a first illumination device 102A and a second illumination device 102B;
b. passing 404 the first and second illuminated light through a first slab 104A and a second slab 104B, respectively;
c. projecting 406 the reflected light on a first zone of interest in a first side 114 of a test strip 106 and a second zone of interest 116 in a second side of the test strip 106 respectively; and
d. capturing 408 one or more images of the first zone of interest disposed on the first side 114 of the test strip and the second zone of interest disposed on the second side 116 of the test strip 106 by way of the image sensor 108.

19. A slab-based optical device 200 comprising:
a. a first illumination device 102A and a second illumination device 102B adapted to provide first luminescence and second luminescence, respectively;
b. a first slab 104A that is disposed below the first illumination device 102A, and is adapted to receive the first luminescence;
c. a second slab 104B that is disposed below the second illumination device 102B, and is adapted to receive the second luminescence;
d. a test strip 106 that is removably disposed between the first slab 104A and the second slab 104B, and adapted to receive the first luminescence and the second luminescence, respectively;
e. a first lens 202 disposed adjacent to an image sensor 108 and adapted to magnify one or more formed images; and
f. the image sensor 108 adapted to capture one or more images of the test strip 106.

20. A slab-based optical device 300 comprising:
a. a first illumination device 102A and a second illumination device 102B adapted to provide first luminescence and second luminescence, respectively;
b. a first slab 104A that is disposed below the first illumination device 102A, and is adapted to receive the first luminescence;
c. a second slab 104B that is disposed below the second illumination device 102B, and is adapted to receive the second luminescence;
d. a test strip 106 that is removably disposed between the first slab 104A and the second slab 104B, and adapted to receive the first luminescence and the second luminescence, respectively;
e. one or more lens 302, 304 disposed between the first slab 104A, second slab 104B and an image sensor 108 and adapted to magnify one or more images formed; and
f. the image sensor 108 adapted to capture one or more images of the test strip 106.
